(19) United States
(12) Patent Application Publication
(10) Pub. No.: US 2007/0088231 A1
(43) Pub. Date: Apr. 19, 2007

(54) Apparatus for axial compression of a patient's spine

(76) Inventor: Daniel S.J. Choy, New York, NY (US)

Correspondence Address: STEVEN L. NICHOLS;RADER, FISHMAN & GRAVER PLLC, 10653 S. RIVER FRONT PARKWAY, SUITE 150, SOUTH JORDAN, UT, 84095, US

(21) Appl. No.: 11/238,918

(22) Filed: Sep. 28, 2005

(51) Int. Cl. A61B 5/103

(52) U.S. Cl. 600/587

(57) ABSTRACT

A device for axial compression of a patient's spine includes a footplate assembly, and a shoulder harness connected to the footplate assembly. The footplate assembly is configured to rest on a stationary surface and to be movable relative to the stationary surface. A method of imaging an intra-disc region includes coupling a shoulder harness to a patient; placing the feet of the patient in contact with a footplate, the footplate being configured to be movable along a stationary surface of an imaging device; establishing a pressure within the intra-disc region by coupling the shoulder harness to the footplate assembly and selectively controlling pressure applied to the coupling between the shoulder harness and the footplate assembly; and imaging the intra-disc region.

### RELATED APPLICATIONS

The present application claims priority under 35 U.S.C. § 119(e) of U.S. Provisional Patent Application No. 60/614,267 filed Sep. 28, 2004, which application is incorporated herein by reference in its entirety.

### BACKGROUND

Human spinal or vertebral column consists of a plurality of separate vertebrae. The vertebrae are joined so as to permit a range of forward, backward, and sideways movement of the column. At the lower end of the spinal column are the lumbar vertebrae, which support the small of the back. Above the lumbar vertebrae are the thoracic vertebrae, which lie behind the thoracic or chest cavity. The uppermost, or cervical, vertebrae define the skeletal framework of the neck. The vertebrae are separated and supported by cartilaginous discs. These discs are subject to deterioration and disease, often creating significant pain. In some cases, the discs rupture or “herniate.”

Studies have shown that the intra-disc pressure in the lumbar spine while in a supine position is about 15 kilo-Pascals (kPa), while pressures in the sitting position average about 150 kPa. Consequently, the observation that patients with herniated lumbar disc disease are often least comfortable in the sitting position may be at least partially due to such pressure differences.

Magnetic Resonance Imaging (MRI) techniques are often used in the diagnosis of lumbar disc disease. Experience has shown, however, that there is often a significant discrepancy between the severity of the patient's clinical symptoms and evidence of disease shown through magnetic resonance imaging. This discrepancy can be explained, in part, by the general inability of conventional MRI systems to allow the patient's spine to be imaged when placed in a variety of positions, including the sitting position, so as to vary the intra-disc pressures and the alignment of the vertebrae.

As indicated above, the supine position produces low intra-disc pressure, e.g., 15 kPa. Almost all magnetic resonance imaging of the lumbosacral spine is performed with the patient in the supine position with consequently low intra-disc pressure. Therefore, disc herniation is less likely to be apparent in the MRI because the patient is in the supine position and experiencing low intra-disc pressure. Consequently, magnetic resonance images taken when the patient is in the supine position can be less than optimal for making an accurate disc herniation diagnosis.

Accordingly, it is extremely useful to produce higher intra-disc pressures in the lumbar region during an MRI procedure. For example, the intra-disc pressure would preferably be on the order of 150 kPa so as to be comparable to the pressure experienced when sitting. To this end, recent efforts have been directed toward creating MRI systems in which the patient sits upright in the machine. However, these MRI systems are rare and expensive. Other efforts have been directed at providing for increased intra-disc pressure in the spine while the patient remains in the supine position required by conventional MRI units. An example of one such device is disclosed in U.S. Pat. No. 6,000,399 issued to Choy, which is incorporated herein by reference in its entirety. This example will be described below.

Referring to FIGS. 1A-1C, a device (10) for creating increased intra-disc pressures while the patient is in a supine position includes a base or frame (12), which may be of rectangular shape as shown in the Figures. This base or frame may be a solid structural member, as shown, or may have an open latticework, a rail-type construction, or the like. The frame is designed to comfortably support a supine patient and is constructed of a material that will not cause false or interfering images during the MRI. For example, wood and plastic structures may be used as the frame (12). The frame is dimensioned to fit within the imaging area of an MRI system.

Shoulder braces (14) are mounted at one end of the frame (12) and spaced apart so that the patient can place his or her head between them while his or her shoulders abut the shoulder braces (14), as shown in FIG. 1C. Each of the shoulder braces (14) may include an upright post (16) to which a cushion (18) is attached. The cushions (18) are oriented such that a patient (36) lying on the frame, as shown in FIG. 1C, can press his or her shoulders against the cushions (18) by apply force with the legs to a foot sled (20) that is secured at the other end of the frame (12).

The location of the foot sled (20) is adjustable so that the sled (20) can be positioned at any number of locations along the frame (12) to best accommodate the height of the patient (36). As best seen in FIG. 1B, the foot sled (20) includes a vertically-extending footboard (22) that is mounted to a horizontal base (24) and braced by diagonal struts (26). The sled may be selectively positioned on the frame (12) by aligning pairs of bores (28) on the sled (20) with corresponding bores (32) in the frame. Pins (34) are then inserted into the aligned bores (28, 32) to secure the position of the foot sled (20) along the length of the frame (12). The shoulder braces (14), the foot sled (20), and the pins (34) are of sufficiently rigid construction to withstand the forces exerted by a patient (36).

As depicted in FIG. 1C, the patient (36) lies supine on the frame (12), his or her shoulders abutting the shoulder braces (14) and his or her feet being placed against the footboard (22) with the knees slightly bent. Typically, the distance between the footboard (22) and shoulder braces (14) should be about three inches less than that of the patient's normal shoulder height.

Once so positioned, the patient (36) exerts pressure with his or her legs, pushes against the footboard (22) and creates pressure on the shoulders which press against the shoulder braces (14). This action also applies a compressive force in the spinal column that can approximate the pressure experienced in a sitting position.

The apparatus (10) and patient (36) are placed in the MRI unit. With the patient (36) exerting pressure with his or her legs to create the desired intra-disc pressure, imaging of the compressed spinal area can be performed. The patient (36) is instructed to maintain the pressure being created by the patient's legs while the MRI scan is performed. The patient can be asked to exert more or less pressure as needed.

A force measuring device, such as a pressure transducer (38), may be mounted to the footboard (22) and connected to a remote display apparatus to provide a quantitative measure of the force being applied by the patient (36). This will also allow the force to be monitored during the duration of a scan to insure relative consistency and to allow suitable instructions to be provided to the patient (36). After the scan is completed, the patient (36) can relax.

This approach creates the desired intra-disc pressure in the spine needed to generate images that are better suited for disc disease diagnosis. However, this approach relies on the patient being physically able to produce, and maintain with relative consistency, intra-disc pressures that are similar to those experienced in a sitting position. This approach also relies on being able to insert the device within the MRI system and relatively position the foot sled and shoulder supports to enable the patient to apply that desired pressure.

Attempts have been made to automate the apparatus in FIGS. 1A to 1C but it was believed that it is necessary that the footplate be securely connected to the flat bed on which the patient was lying. See U.S. Pat. No. 5,779,733. This resulted in a cumbersome apparatus that was difficult to position on an MRI machine. The present invention advantageously eliminates any such connection, and therefore provides a more flexible and more easily mountable structure.

### SUMMARY

A device for axial compression of a patient's spine includes a footplate assembly, and a shoulder harness connected to the footplate assembly. The footplate assembly is configured to rest on a stationary surface and to be movable relative to the stationary surface. A method of imaging an intra-disc region, includes coupling a shoulder harness to a patient; placing feet of the patient in contact with a footplate, the footplate being configured to rest on a stationary surface of an imaging device; establishing a pressure within the intra-disc region by, coupling the shoulder harness to the footplate assembly, and selectively controlling pressure applied to the coupling between the shoulder harness and the footplate assembly; and imaging the intra-disc region.

### BRIEF DESCRIPTION OF THE DRAWINGS

The accompanying drawings illustrate various embodiments of the present apparatus and method and are a part of the specification. The illustrated embodiments are merely examples of the present apparatus and method and do not limit the scope of the disclosure.

FIGS. 1A-1C illustrate a prior art apparatus.

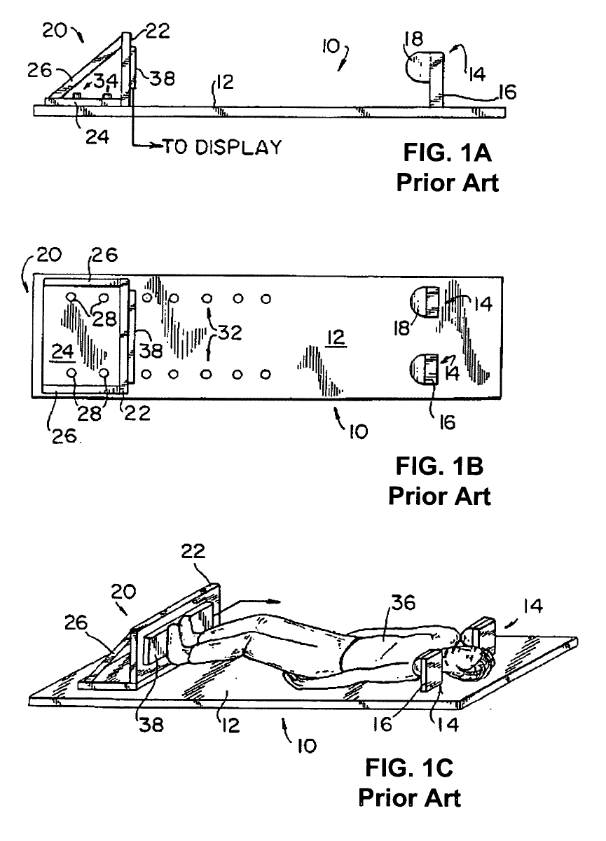

FIG. 2 illustrates a device for applying and controlling intra-disc pressures in a patient's spine according to one exemplary embodiment.

FIG. 3 illustrates a patient using the device of FIG. 2.

FIG. 4 illustrates a footplate assembly according to one exemplary embodiment.

FIG. 5 is a graph showing the correlation between footplate pressure in pounds-per-square-inch (PSI) and intra-disc pressure in kilo-Pascals (kPa).

FIG. 6 illustrates the device and patient being placed within an MRI system.

FIG. 7 illustrates a method of using the device to establish pressure in the lumbar discs according to one exemplary embodiment.

Throughout the drawings, identical reference numbers designate similar, but not necessarily identical, elements.

### DETAILED DESCRIPTION

Systems and methods are described herein for selectively establishing pressure in the spine of a patient, particularly in the lumbar disc region, while the patient is in a supine position. The systems and methods disclosed make use of a footplate assembly and shoulder harness that are not attached to a frame or stationary surface. Such a configuration may provide for a relatively simple imaging operation and a relatively simple device for establishing a pressure while the patient is placed within an imaging system. Further, the systems and methods are provided herein for directly reading the intra-disc pressure established in the lumbar disc region of the patient. Such readings are provided in kilo-Pascals.

In the following description, for purposes of explanation, numerous specific details are set forth in order to provide a thorough understanding of the present method and apparatus. It will be apparent, however, to one skilled in the art that the present method and apparatus may be practiced without these specific details. Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment.

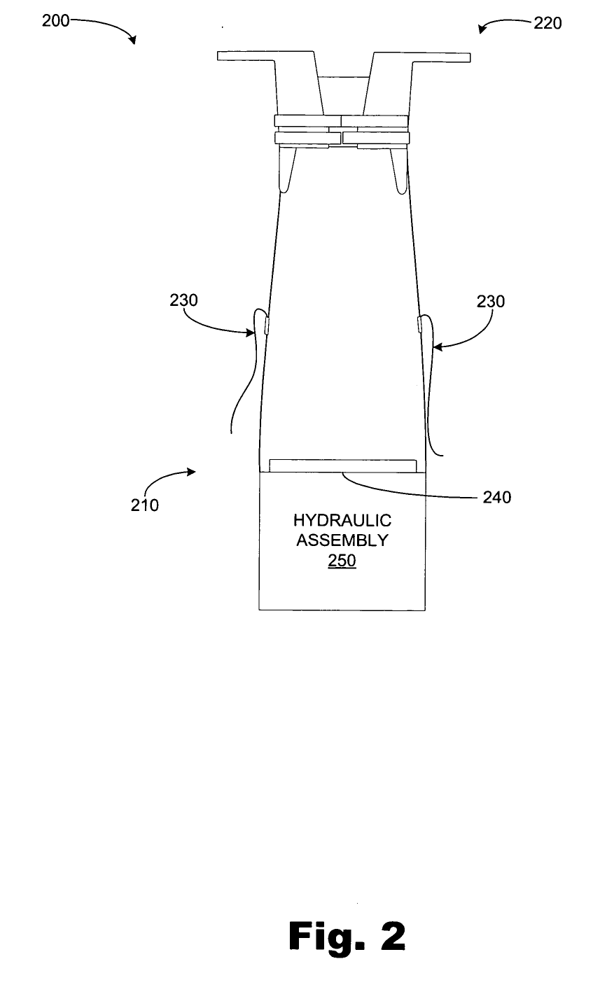

FIG. 2 illustrates an example of a device (200) for creating intra-disc pressure within the human spine. The device (200) generally includes a footplate assembly (210) coupled to a shoulder harness (220) by a number of straps (230). The device (200) is configured to exert controllably increased pressure in, for example, the lumbar discs of a patient, when the patient is in a supine position. This increase in pressure is of a similar magnitude to the pressure that would be experienced if the patient were in a sitting position.

The footplate assembly (210) generally includes a footboard (240) and a hydraulic assembly (250). The footboard (240) is not attached to any frame or support surface, such as the surface of an MRI system. The hydraulic assembly (250) controls the forced exerted by the straps (230) between the shoulder harness (220) and the footboard (240), and the consequent pressure applied to a patient. The device (200) will also provide a pressure reading of the intra-disc pressure being created.

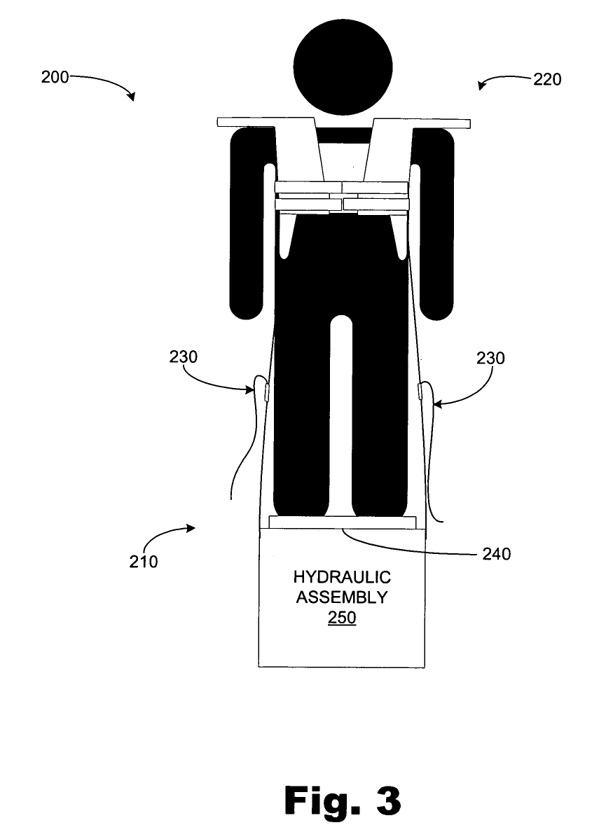

FIG. 3 shows the device (200) in use by a patient. As seen in FIG. 3, the shoulder harness (220) is worn on the shoulders of the patient. The shoulder harness (220) is adjustable so that it can be fitted to the patient. The shoulder harness (220) may be secured to the patient in a variety of ways, for example, hook and loop straps, buckles, ties, buttons, clasps and the like.

As will be described in detail below, the device (200) provides compressive forces, which are transmitted through the patient's skeletal structure to the spine, particularly the lumbar spine area. The patient may be positioned within an MRI system before the device (200) is worn or activated to obtain an image of the lumbar disc region in an uncompressed state.

For example, the shoulder harness (220) may be placed on the patient while the patient is outside of the MRI system. Thereafter, the patient is placed within the MRI system while wearing the shoulder harness (220). At that point, an image of the lumbar disc region in an uncompressed state is taken.

Thereafter, the footboard (240), which is portable, is placed in contact with the feet of the patient. The straps (230) are then connected between the harness (220) and the footplate assembly (210). The straps (230) are adjusted so as to be taut between the harness (220) and footplate assembly (210). The hydraulic unit (250) is then used to apply pressure on the straps (230), pulling the harness (220) toward the footboard (240). If the patient does not bend his or her knees in response to this pressure, the pressure will be transmitted through the patient's skeletal structure and create an elevated intra-disc pressure in the spine.

After imaging is complete, the hydraulic unit (250) is also configured to release the pressure on the straps (230), thereby decreasing the pressure applied by the device (200) to the patient. The hydraulic unit (250) may release the pressure automatically if the pressure exceeds a maximum threshold.

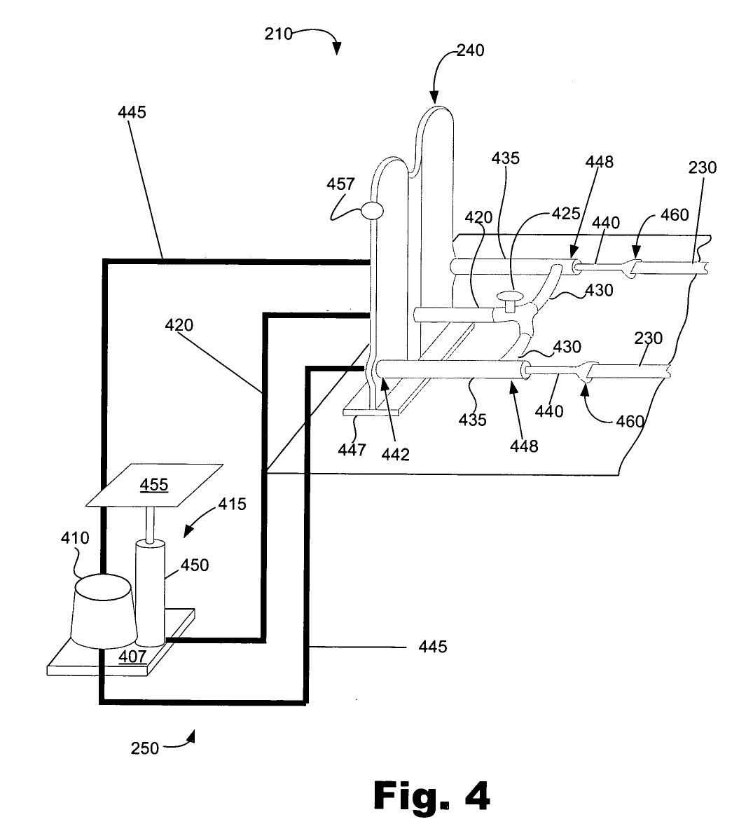

One exemplary footplate assembly and hydraulic unit will now be discussed in detail. FIG. 4 illustrates a footplate assembly (210) according to one exemplary embodiment. The footplate assembly (210) generally includes a hydraulic assembly (250) and a footboard (240).

The footboard (240) is coupled to a platform base (447) so that the footboard (240) can be maintained with stability in an upright position. The platform base (447) is configured to rest on a stationary surface while allowing the footboard (240) to remain oriented substantially normal to that surface on which the platform base (447) rests. Such a configuration allows the footboard (240) to be moved as desired, rather than being secured to a stationary surface or frame.

The hydraulic assembly (250) includes, according to one exemplary embodiment, a base (407) that supports a flexible reservoir (410) and a pump unit (415). The pump unit (415) may be a hand or foot pump. In the illustrated example, the pump (415) is a foot pump including a pedal (455) for driving a plunger in a cylinder (450). A hydraulic line (420) is coupled between the pump (415) and a valve (425). The valve (425) controls the direction of flow from the pump unit (415) to the rest of the hydraulic assembly (250). In particular, when the valve (425) is in a first position, liquid is allowed to flow from the pump unit (415), through the valve (425), and to first and second interconnects (430). In this first position, the valve (425) also prevents liquid from flowing back to the pump unit (415).

The interconnects (430) are coupled respectively to two hydraulic cylinders (435). Pistons (440) in the cylinders (435) are attached to the straps (230) that run to the shoulder harness. The interconnects (430) provide fluid from the pump unit (415) into the distal ends (448) of the hydraulic cylinders (435). Fluid flowing from the pump unit (415) and into the distal ends (448) of the cylinders (435) compresses the pistons (440) in the cylinders thereby exerting a pull or pressure on the straps (230) connected to the pistons (440). This pressure is transmitted by the straps (230) to the shoulder harness and the patient.

Return lines (445) allow fluid in the other or proximal ends (442) of the cylinders (435) to flow into the flexible reservoir (410). This occurs as the pistons in the cylinders (435) are compressed and force fluid into the return lines (445) from the proximal ends (442) of the cylinders (435).

The hydraulic line (420) and return lines (445) may be flexible, such that the footboard (240) may be moved relative to the pump unit (415), the flexible reservoir (410), and the support plate (407). This facilitates placing the patient in the MRI system and connecting the footboard (240), via the straps (230), to the shoulder harness on the patient.

The pump unit (415) and the flexible reservoir (410) may be located below the surface supporting the footboard (240). This is advantageous where, as here, the pump unit (415) is a foot pump.

As described above, the pump unit (415) includes a cylinder (450) with a plunger and foot pedal (455). The pump unit (415) may include a biasing member to drive the plunger and pedal (455) upward. As the plunger and pedal (455) are urged or drawn upward, the pump unit (415) draws liquid from the reservoir (410). The foot pedal (455) provides a platform for the operator to exert pressure on the pump (415). As the pedal (455) is depressed, liquid is forced from the pump unit (415) and into the hydraulic line (420). The liquid flows through the hydraulic line (420) until it comes to the valve (425). If the valve (425) is in the first position described above, the liquid flows through the valve (425) and interconnects (430) into the distal ends (448) of the hydraulic cylinders (435). Consequently, the pistons (440) are compressed and pressure is applied to the straps (230).

The pump unit (415) may be operated through a series of strokes to obtain the desired pressure on the patient's spine. Once the desired pressure is achieved, the valve (425) is closed to maintain the desired hydraulic pressure within the system. The hydraulic unit is adjusted to establish a target intra-disc pressure of approximately 150 kPa. 10-15 kPa more or less than 150 kPa will still enable the system to obtain useful imaging of the lumbar disc region under pressure.

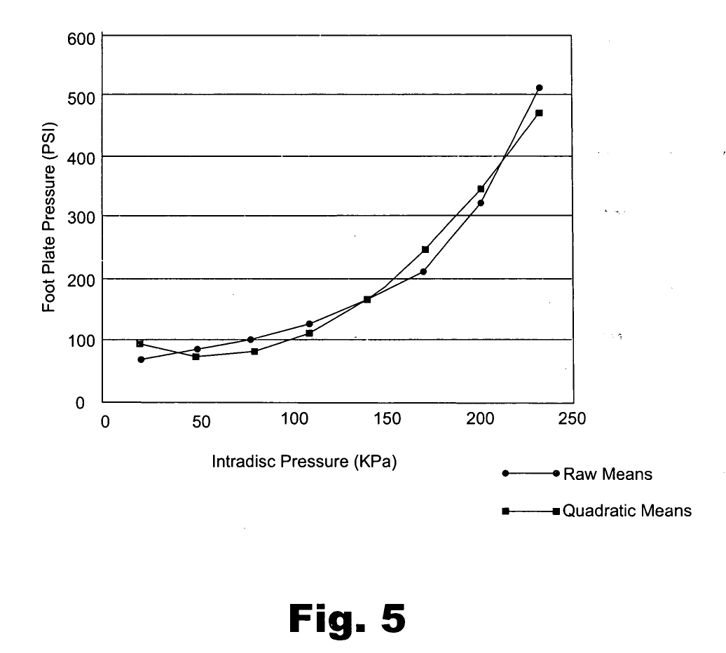

To determine when the desired intra-disc pressure has been achieved, a pressure gauge (457) is coupled to the footboard (240). It has been discovered that a measure of the pressure of a patient's feet on the footboard (240), as measured, for example, in pounds-per-square-inch (PSI) is directly correlated to intra-disc pressure, measured in kPa. This correlation is illustrated in FIG. 5 and has been established by actually measuring and comparing the pressure at the footboard with intra-disc pressure in a number of patients over a range of applied pressures. Interestingly, the correlation illustrated in FIG. 5 holds true regardless of the patient's size, weight or body shape.

Using the correlation in FIG. 5, the pressure gauge (457) can be calibrated to measure the pressure at the footboard (240) and output a direct reading of intra-disc pressure in the spine in kilo-Pascals. This is extremely helpful for establishing the desired intra-disc pressure needed for accurate imaging and the diagnosis of lumbar disc disease.

As a safety feature, the pressure gauge (457) is configured to release the pressure in the hydraulic assembly (250) when the intra-disc pressure reaches a maximum level, such as 200 kPa. For example, the gauge (457) may close the valve (425) or other liquid pathway to prevent further pressure from being built up. The gauge (457) may also adjust the valve (435) to allow fluid to flow out of the distal ends (448) of the cylinders (435) and back into the hydraulic line (420) to ease pressure on the straps (230). The gauge (457) may also divert further fluid to other parts of the hydraulic assembly (250) to reduce the pressure on the patient.

After imaging, the pressure applied to the patient by the system is released. The hydraulic assembly (250) is configured to release the pressure driving the ends of the straps (230) toward the footboard (240). According to the present exemplary embodiment, the valve (425) is configured to be selectively turned to a second position. While in the second position, the valve (425) allows liquid to flow from the interconnects (435) back through the hydraulic line (420). Compression of the flexible reservoir (420) drives fluid back through the return lines (445) and into the proximal ends (442) of the cylinders (435). This drives the pistons (440) away from the footboard (240) and releases the pressure in the straps (230). The movement of the pistons (440) drives fluid from the distal ends (448) of the cylinders (435), through the interconnects (435) and back into the hydraulic line (420).

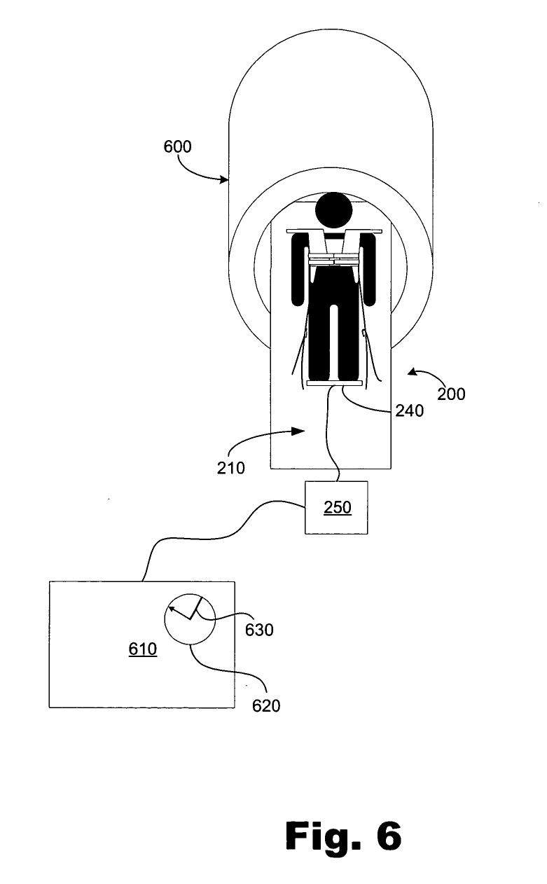

As shown in FIG. 6, the configuration of the device (200) allows the patient to be placed within an MRI system (600) while the footplate assembly (210) remains outside. As a result, the footplate assembly (210) allows for control of pressure in the lumbar discs of the patient while the patient is within the MRI system (600).

The gauge (457; FIG. 4) of the footplate assembly (210) senses the amount of pressure applied to the footboard (240). This pressure determination can also be output to and displayed on a remote gauge (620) that is part of a control console (610). This allows an MRI system operator who is located at the console (610) to monitor the pressure applied by the device (200) to ensure that the pressure is adequate, consistent and within the desire range. As indicated above the pressure reading on the remote gauge (620) represents the intra-disc pressure the patient is experiencing in kilo-Pascals. The remote gauge (620) can also send a signal to a computer or other processing device of the control console (610) indicating the pressure applied. This computer or other processing device may be recording the pressure levels during imaging, operating a safety system that releases the pressure applied by the system if that pressure exceeds a maximum threshold, etc.

Further, the remote gauge (620) may include an indicator (630) that corresponds to a desired or target lumbar disc pressure. The desired or target lumbar disc pressure is 150 kPa. The indicator (630) may also show a maximum pressure, such as 200 kPa, that should not be exceeded.

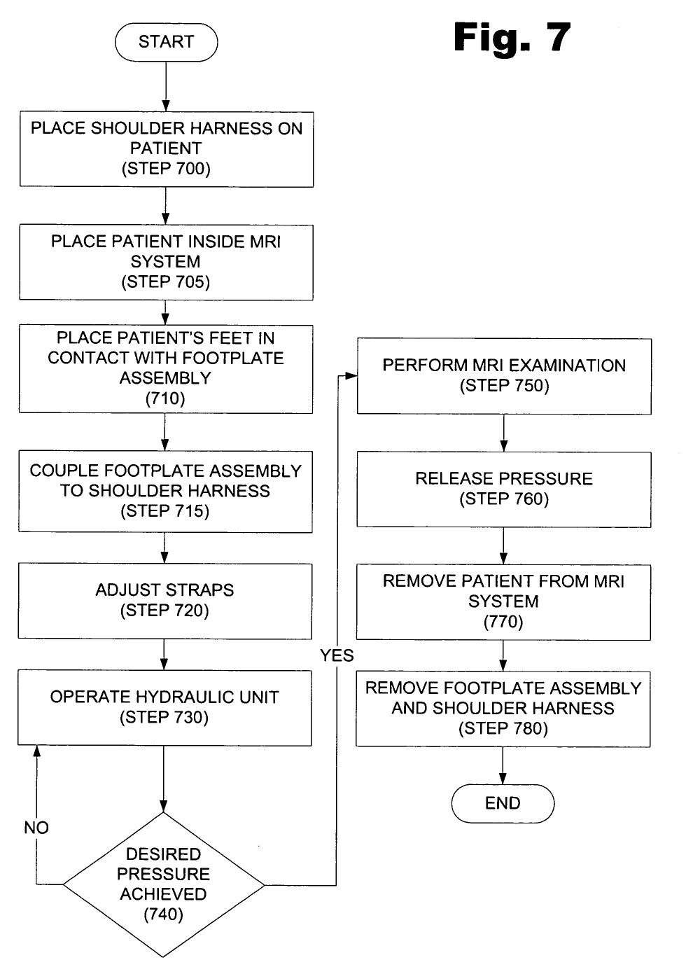

FIG. 7 illustrates a method of operating the devices described herein to obtain an image of a patient's spine under a desired pressure. As shown in FIG. 7, the method begins by placing the shoulder harness on the patient (step 700). As indicated above, the harness can be adjusted to comfortably fit the patient. The harness has a structure such that when force is applied to the straps, the harness will evenly distribute the pressure across the patient's shoulders and, through the patient's skeletal structure, to the patient's spine, particularly the lumbar region.

Next, the patient, wearing the shoulder harness, is placed in the MRI system (step 705). The footplate assembly is then put in place with the patient's feet against the footboard (step 710). The footplate assembly is then connected by the straps or other means to the shoulder harness (step 715). The straps are adjusted to be taut (step 720).

Then, the hydraulic unit is operated, as described above, to apply pressure to the patient through the straps and shoulder harness (step 730). Using the gauge described above, the pressure applied is monitored until the desired pressure is achieved (determination 740) e.g., an intra-disc pressure of approximately 150 kPa.

When the desired pressure has been achieved, the magnetic resonance imaging is performed to obtain the desired images of the patient's spine under the applied pressure (step 750). When imaging is completed, the pressure is released (step 760) and the patient is removed from the MRI system (step 770). The footplate assembly and shoulder harness are removed (step 780).

The preceding description has been presented only to illustrate and describe the present method and apparatus. It is not intended to be exhaustive or to limit the disclosure to any precise form disclosed. Many modifications and variations are possible in light of the above teaching. It is intended that the scope of the invention be defined by the following claims.

What is claimed is:

1. A device for axial compression of a patient's spine, comprising:
a footplate assembly; and
a shoulder harness connected to said footplate assembly,
wherein said footplate assembly is configured to rest on a stationary surface and to be movable relative to said stationary surface.

2. The device of claim 1, wherein said footplate assembly is connected only to said shoulder harness and not to said stationary surface.

3. The device of claim 1, wherein said footplate assembly comprises a hydraulic unit for applying pressure to said connection between said footplate assembly and said shoulder harness

4. The device of claim 3, wherein said footplate assembly and shoulder harness are connected with non-distensible straps

5. The device of claim 3, further comprising a gauge coupled to said footplate assembly for measuring a pressure exerted on said footplate assembly.

6. The device of claim 5, wherein said gauge outputs an indication of intra-disc pressure experienced by a patient based on a correlation of said pressure exerted on said footplate assembly and said intra-disc pressure.

7. The device of claim 5, wherein said gauge is located at a remote location away from said footplate assembly.

8. The device of claim 3, wherein said hydraulic unit comprises a flexible reservoir and a pump unit.

9. The device of claim 8, wherein said hydraulic unit is configured to be foot-actuated.

10. The device of claim 8, wherein said hydraulic unit further comprises at least one hydraulic cylinder and at least one piston, wherein movement of said piston applies pressure to said connection between said shoulder harness and said footplate assembly.

11. The device of claim 10, wherein said hydraulic unit comprises a valve for regulating the pressure on said connection.

12. A method of imaging an intra-disc region, comprising:
coupling a shoulder harness to a patient;
placing the feet of said patient in contact with a footplate, said footplate being configured to be movable along a stationary surface of an imaging device;
establishing a pressure within said intra-disc region by coupling said shoulder harness to said footplate assembly and selectively controlling pressure applied to said coupling between said shoulder harness and said footplate assembly; and
imaging said intra-disc region.

13. The method of claim 12, and further comprising performing a preliminary imaging operation on said intra-disc region before applying said pressure.

14. The method of claim 12, wherein imaging said intra-disc region includes performing a magnetic resonance imaging operation.

15. The method of claim 12, wherein establishing said pressure includes establishing a pressure of about 150 kPa.

16. The method of claim 15, wherein establishing said pressure includes monitoring a gauge, said gauge being configured to display an intra-disc pressure value based on a correlation between pressure on said footplate assembly and intra-disc pressure.

17. The method of claim 16, wherein monitoring said gauge includes monitoring intra-disc pressure values in kPa.

18. The method of claim 12, further comprising releasing said pressure if said pressure reaches a maximum threshold.

19. A method of monitoring intra-disc pressure in a patient, said method comprising:
measuring pressure applied by said patient's feet to a footboard;
correlating said pressure with intra-disc pressure experienced by said patient; and
outputting an indication of intra-disc pressure.

20. The method of claim 19, further comprising applying pressure with a shoulder harness worn by said patient and attached to said footboard to generate said pressure applied by said patient's feet to said footboard.
